(19) United States
(12) Patent Application Publication
(10) Pub. No.: US 2024/0344086 A1
(43) Pub. Date: Oct. 17, 2024

(54) COMPOSITION AND METHODS FOR RECOMBINANT LENTIVIRAL PRODUCTION

(71) Applicant: AMGEN INC., Thousand Oaks, CA (US)

(72) Inventors: Christoph Adrian Kahl, Thousand Oaks, CA (US); Haejin Kim, Newbury Park, CA (US)

(21) Appl. No.: 18/701,723

(22) PCT Filed: Oct. 19, 2022
(86) PCT No.: PCT/US22/78377

§ 371 Date: Apr. 16, 2024

Related U.S. Application Data
(60) Provisional application No. 63/257,479, filed on Oct. 19, 2021.

(51) Int. Cl. C12N 15/86

(52) U.S. Cl. CPC C12N 15/86; C12N 2740/16022; C12N 2740/16043; C12N 2740/16052; C12N 2760/20243

(57) ABSTRACT

Disclosed herein are lentiviral plasmid packaging compositions comprising unique mass ratios of four plasmids that are useful in producing lentiviral vectors. Also provided are methods of producing such lentiviral vectors by mixing a complexation solution comprising the lentiviral plasmid packaging composition with a transfection reagent and incubating the mixture for at least 10 minutes. transfecting a eukaryotic host cell with the lentiviral plasmid packaging composition, adding sodium butyrate to the transfected host cells about one day after transfection, and culturing the host cell. Exemplary embodiments of the method comprise a concentration of total plasmid in pg/mL of 0.25-3 and/or a mass ratio of total plasmid mass in pg to the mass of transfection reagent in pg extending from about 1:1 to about 1:3.

### CROSS-REFERENCE TO RELATED APPLICATIONS

This application claims priority to U.S. Provisional Patent Application No. 63/257,479, filed Oct. 19, 2021, which is hereby incorporated by reference in its entirety.

### Incorporation by Reference of the Sequence Listing

This application contains, as a separate part of disclosure, a Sequence Listing in computer-readable form (Filename: 56742_Seqlisting.XML; 40, 107 bytes—ASCII text file dated Oct. 19, 2022) which is incorporated by reference herein in its entirety.

### FIELD

The disclosure relates to compositions and methods of producing lentiviral vectors comprising at least one nucleic acid for use in clinical applications for genetic modification of cells to treat diseases and disorders.

### BACKGROUND

Several techniques for introducing exogenous nucleic acid into a cell have been developed over the past several decades, with those techniques generally grouping into chemical, electrical or vector-driven biological methodologies to mitigate or overcome the rapid degradation that occurs upon entry of naked nucleic acid into a cell. For introduction of nucleic acids into cell In vivo, such as in gene therapy, biological methodologies are the methods of choice.

A variety of vectors have been employed in delivering nucleic acids to cell in vivo, but viruses stand out as providing vectors with a range of cell tropisms, controllable expression of the exogenous nucleic acid payload, and persistence, leading to the potential for long-term expression of exogenous nucleic acid at appropriate levels. Within the class of viral vectors, retroviruses and, more particularly, lentiviruses stand out as providing a desirable set of the aforementioned properties. The general class of retroviruses provide the advantages of their wide cell tropism and because they efficiently integrate into the host cell genome, leading to long-term expression of exogenous nucleic acid payloads. Some members of the retroviral class of vectors do suffer from limitations, however. Retroviruses generally do not efficiently infect quiescent cells, exhibiting a narrowed cell tropism that can be problematic in using such techniques as gene therapy to treat disease. Unlike other retroviruses, however, the lentiviruses (e.g., HIV-1) do efficiently infect non-dividing (quiescent or terminally differentiated cells) as well as dividing cells. These advantages have led to a focus on lentiviral vector development to maximize their capacity to deliver exogenous nucleic acid to a wide variety of cells in a safe manner.

Three different generations of lentiviral vectors have been established, with safety increasing in each generation. The first generation of lentiviral vectors involved a system consisting of three plasmids. A first plasmid encoded elements of the lentiviral vector genome, including the wild-type 5′ and 3′ Long Terminal Repeats (LTRs), the Psi (ψ) sequence, a fragment of the env gene containing the Rev Response Element (RRE), an internal promoter, and the exogenous nucleic acid (e.g., desired gene). A second plasmid provided the components for packaging recombinant lentiviruses by containing the HIV-1 genome containing all viral genes with the exception of the env gene. A third plasmid defined the wide cell tropism of the recombinant lentivirus by encoding the vesicular stomatitis virus G protein (VSV-G). This system, however, posed a reduced but nonetheless still unacceptable, risk of producing replication competent lentiviruses (RCL) in vivo.

The second-generation lentiviral vector system differed from the first-generation system by eliminating HIV accessory proteins not essential to the production of the lentiviral particle. This second-generation system is safer than the first generation. By eliminating non-essential HIV coding regions, the risk of generating RCL is lowered relative to wild-type lentivirus and the first-generation lentiviral vector system, but some risk remains, particularly if the exogenous nucleic acid encodes a proto-oncogene or the HIV status of the patient to be treated is unknown.

The third-generation lentiviral vector system was developed for safe use in the treatment of disease, such as the treatment of human patients. In this generation of the lentiviral vector system, the HIV tat gene is no longer present. The tat gene is involved in driving expression of exogenous nucleic acid from the lentiviral LTRs. In addition, Rev, which facilitates nuclear export of expressed gene product, is expressed from a separate plasmid, and the promoter of the 5′ LTR has been deleted to reduce its activity. A Cytomegalovirus (CMV) or an Elongation Factor 1α (EF1α) promoter is inserted in the 5′ LTR lacking its native promoter, eliminating the need for Tat to transcribe the viral genome. Currently, the third-generation lentiviral vector system offers the best safety profile in terms of reduced RCL generation because this vector system requires only three HIV-1 genes (gag, pol, and rev) for production. Improving the safety of these vector systems is still an active area of research due to the possibility that mutation or recombination with human retroviruses could lead to RCL.

Lentiviral vectors have become a gene transfer system of choice for gene and cell therapy applications, in particular in the field of oncology to transfer T-Cell Receptor (TCR) or Chimeric Antigen Receptor (CAR), thus generating armed T cells capable of locating and destroying cancer cells. In order to improve the therapeutic efficacy, immunomodulatory genes, such as Interleukins (ILs), as well as other expressed or regulatory elements can be added to the heterologous gene cassette, resulting in total proviral lengths close to, or even above, the size of wild-type lentivirus genomes (about 9-10 kb). It is known in the art, however, that packaging of large transgene cassettes leads to a marked decrease in vector titers. Kumar et al., Hum Gene Ther. 12(15):1893-905 (2001) showed that lentiviral vectors can package a wide range of insert sizes, including inserts leading to oversized provirus length, thus establishing that lentiviral vectors do not have a hard packaging limit. Vector titers are most optimal with undersized genomes, however, and titers decrease in a semi-logarithmic fashion with increasing proviral length above about 5 kb. The developing field has focused on optimizing lentiviral vector production using small and simple transgene cassettes, typically expressing a marker gene, such as green fluorescent protein (GFP). The current production methods described in WO 2018/064584 A1, WO 2017/091786 A1, EP 3 327 119 A1 solely address small heterologous inserts (up to 2 kb). Therefore, methods for packaging medium (about 2-4 kb) and complex large (greater than 4 kb) multigene cassettes to meet rising clinical demand remains a need in the art.

Commonly used approaches to increase lentiviral vector titers rely on addition of animal-derived components to the media to improve cell growth and viral vector productivity, such as Dulbecco's Modified Eagle Medium (DMEM) containing fetal bovine serum (FBS), and to solutions used for transfection complexation, such as Opti-MEM containing human-derived transferrin (Thermofisher Scientific catalog no. A4124801). These animal-derived components introduce complexity, cost, and variability into the vector production process, and also pose significant safety and regulatory concerns for the development of clinical therapeutics. Other common ways to increase viral vector titer exist in the field, though typically resulting in clinical safety or manufacturing cost concerns. In particular, HEK293T cell lines can boost lentiviral vector titers compared to HEK293 cells, as described in Ausubel et al., Bioprocess Intl. 10(2):32-43 (2012). The HEK293T cell line, however, contains the large T antigen from simian vacuolating virus 40 (SV40), a known oncogene that poses significant safety concerns for clinical use. Thus, a need continues to exist in the art for methods of lentiviral vector production designed for use in clinical applications by avoiding animal-derived components or cell lines expressing oncogenes.

In addition, a method described by al Yacoub et al., J Gene Med. 9(7):579-84 (2007) improved lentivector titers, but only by performing transient plasmid transfection in adherent cell cultures with 12.4 μg DNA per 10⁶ cells. The methods disclosed by al Yacoub et al. highlight needs in the art for LVV production methods suitable for transient plasmid transfection of suspension cell cultures and capable of achieving effective transfection levels using lower concentrations of DNA per quantity of cells.

Current lentiviral vector productions methods solely address the optimization of small to medium-sized transgene cassettes (i.e., heterologous inserts of less than about 4 kb). We demonstrate herein that such methods do not sufficiently rescue the low titer of lentiviral vectors that carry clinically relevant large and complex transgene cassettes. Therefore, both the need for packaging large transgenes and rising clinical demand necessitate the development of an optimized production method as described herein.

### SUMMARY

The compositions and methods disclosed herein provide an approach to lentiviral vector production that is completely free of animal or human components. The disclosure provides a lentiviral vector (LVV) composition and production method that benefit from the surprising discovery of particular mass ratios of LVV plasmids to maintain high titers of infectious, functional recombinant LVVs containing large (greater than 4 kb) nucleic acid inserts as genes of interest (GOI), such as transgenes encoding T-Cell Receptors (TCRs) or Chimeric Antigen Receptors (CARs), or nucleic inserts comprising multiple coding regions or transgenes collectively characterized as large nucleic acid inserts or GOIs. The large GOI(s) are greater than 4 kb, such as a GOI that is greater than 4 kb up to 8 kb or greater than 4 kb up to 7.5 kb. In contrast to the LVV production methods currently in use, which do not sufficiently rescue the low titer of lentiviral vectors that carry clinically relevant medium to large and complex transgene cassettes (greater than 2 kb), the disclosed methods produce useful titers of LVV carrying medium to large, complex and/or multiple gene or coding region cassettes. In providing production methods for LVV comprising these relatively large GOI(s), the disclosure provides methods of producing significant titers of LVV comprising the relatively large GOI(s). Moreover, the disclosed methods of production can achieve the usefully high LVV titers using an animal-free production method, facilitating the production of a new category of therapeutics. The methods disclosed herein provide an approach to lentiviral vector transient transfection that is completely free of commonly used animal or human components such as serum, transferrin, and the SV40 large T antigen. Therefore, the disclosed methods provide both a better clinical safety profile and a simplified manufacturing process. As used herein, the term “animal-free” is used to denote that a transfection reagent or process does not utilize any material derived from animals or humans, whereas “xeno-free” denotes the absence of animal, but not human-derived material. In contrast to state-of-the-art LVV production methods such as the methods of al Yacoub et al. (2012), the methods disclosed herein permit transfection in both suspension culture and at 1.5 μg DNA/mL culture medium at a cell density of greater than 5×10⁶ cells/mL, thereby achieving substantial plasmid savings.

In one aspect, the disclosure provides a lentiviral plasmid packaging composition comprising a gag/pol plasmid, a rev plasmid, an envelope plasmid, and a transfer plasmid, wherein the plasmids are present in a mass ratio of 1.1 gag/pol plasmid:1 rev plasmid:1.1 envelope plasmid:3.2 transfer plasmid, wherein the plasmid packaging composition lacks a wild-type lentiviral 5′ long terminal repeat (LTR) promoter, and wherein the transfer plasmid comprises a heterologous nucleic acid greater than 2 kb. In some embodiments, the envelope plasmid comprises a coding region for vesicular stomatitis virus G protein (VSV-G). In some embodiments, the heterologous nucleic acid is greater than 4 kb, which includes embodiments in which the heterologous nucleic acid is at least 5 kb, is 4-8 kb, is 4-7.5 kb, is 4-5 kb, or is greater than 8 kb.

Some embodiments of this aspect of the disclosure provide a lentiviral plasmid packaging composition as described herein wherein the heterologous nucleic acid encodes a T-cell receptor, a chimeric antigen receptor, or a multi-gene complex that may comprise one or more structural genes and may further comprise one or more regulatory elements typically involved in controlling the expression of at least one of the structural genes. In some embodiments, the total DNA concentration of gag/pol plasmid, rev plasmid, envelope plasmid, and transfer plasmid is 0.25-2.5 μg/mL, including embodiments wherein the total DNA concentration of gag/pol plasmid, rev plasmid, envelope plasmid, and transfer plasmid is 1.0-2.0 μg/mL, e.g., 1.0-1.5 μg/mL. In some embodiments, the lentiviral plasmid packaging composition lacks a lentiviral tat gene. In some embodiments, the lentiviral plasmid packaging composition further comprises a complexation solution, such as OPTI-MEM, Opti-plex, FreeStyle™, or LV-MAX. In some embodiments, the lentiviral plasmid packaging composition further comprises a transfection reagent, including embodiments wherein the transfection reagent is PEIpro or PEI-MAX.

Some embodiments of the lentiviral plasmid packaging composition comprise a total plasmid concentration in the range of 0.25-3.5 μg/mL, such as a concentration range of 0.25-2.5 μg/mL or 1-3 μg/mL. The total plasmid concentration refers to the concentration of plasmid in the total production volume (LVV production scale), which includes the cells to be, or being, transfected. This range of total plasmid concentrations yielded high transfection efficiencies.

Another factor relevant to the production of lentiviral plasmid packaging compositions according to the disclosure is the mass ratio of total plasmid to transfection reagent (e.g., PEI-MAX, PEIpro). An exemplary mass ratio of total plasmid to transfection reagent is 1 μg total plasmid:3 μg transfection reagent.

In some embodiments, the gag/pol plasmid comprises the polynucleotide sequence set forth at SEQ ID NO: 1, which contains coding regions for expressing the following amino acid sequences: Gag (SEQ ID NO:2) and Pol (SEQ ID NO:3). The gag/pol plasmid also comprises a coding region for an ampicillin resistance marker. The rev plasmid comprises the polynucleotide sequence set forth at SEQ ID NO:4, which contains coding regions for expressing the following amino acid sequences: Rev (SEQ ID NO:5) and Ampicillin resistance (SEQ ID NO:6); and the envelope plasmid comprises the polynucleotide sequence set forth at SEQ ID NO:7, which contains a coding region for expressing the amino acid sequence of VSV-G (SEQ ID NO:8). The envelope plasmid also comprises a coding region for an ampicilin resistance marker.

Another aspect of the disclosure is drawn to a method of producing a recombinant lentiviral vector comprising: (a) preparing a mixture comprising the lentiviral plasmid packaging composition disclosed herein and a transfection reagent in a ratio of 1 total plasmid concentration in Σg/mL:greater than 1 μg/mL total transfection reagent; (b) infecting a eukaryotic host cell with the mixture; and (c) culturing the infected host cell, thereby producing the recombinant lentiviral vector. In some embodiments, the mixture comprising a complexation solution of the lentiviral plasmid packaging composition described herein and the transfection reagent is incubated for at least 10 minutes before infecting the eukaryotic host cell. In some embodiments, the method further comprises adding sodium butyrate, e.g., 10 mM sodium butyrate, to the infected host cell culture about one day after infection. In some embodiments, the envelope plasmid comprises a coding region for vesicular stomatitis virus G protein (VSV-G). In some embodiments, the heterologous nucleic acid is at least 2 kb, including embodiments in which the heterologous nucleic acid is at least 4 kb, is at least 5 kb, is 4-8 kb, is 4-7.5 kb, is 4-5 kb, or is greater than 8 kb.

In some embodiments of the method, the heterologous nucleic acid encodes a T-cell receptor (i.e., TCR), a chimeric antigen receptor (i.e., CAR), or a multi-gene complex that may comprise one or more structural genes and may further comprise one or more regulatory elements typically involved in controlling the expression of at least one of the structural genes. such as a clinically relevant set of structural gene(s) and regulatory element(s). Exemplary multi-gene constructs include clinically relevant sets of structural of structural gene(s) and regulatory element(s), such as a TCR and an Interleukin gene or a CAR and an Interleukin gene, with or without regulatory element(s) controlling the expression of these genes or gene constructs. In some embodiments of the method, the lentiviral packaging composition lacks a lentiviral tat gene. In some embodiments, the method further comprises a complexation solution, such as wherein the complexation solution is OPTI-MEM or Opti-plex. In some embodiments, the method further comprises a transfection reagent, such as wherein the transfection reagent is PEIpro or PEI-MAX. In some embodiments of the method, the concentration of total plasmid in μg/mL is 0.25-3.5, such as a concentration range of 0.25-3.0, 0.25-2.5, 1-3, or 1-2. Additionally, the mass ratio of total plasmid mass to mass of transfection reagent is another consideration in producing the lentiviral packaging compositions according to the disclosure, as noted above. An exemplary mass ratio of total plasmid mass to transfection reagent mass is 1 μg total plasmid:3 μg transfection reagent (e.g., PEI-MAX, PEIpro). In some embodiments, the total DNA concentration of gag/pol plasmid, rev plasmid, envelope plasmid, and transfer plasmid is 0.25-3.5 μg/mL, 0.25, 3.0, 0.25-2.5 μg/mL, 1.0-3.0, or 1.0-2.0 μg/mL. In some embodiments, the recombinant lentiviral vector is harvested at day 2-3 post-transfection, such as wherein the recombinant lentiviral vector is harvested at day 2 post-transfection. In some embodiments of the method, the gag/pol plasmid comprises the sequence set forth at SEQ ID NO: 1, the rev plasmid comprises the sequence set forth at SEQ ID NO:4, and the envelope plasmid comprises the sequence set forth at SEQ ID NO:7.

Other features and advantages of the disclosure will become apparent from the following detailed description, including the drawings. It should be understood, however, that the detailed description and the specific examples, while indicating preferred embodiments, are provided for illustration only, because various changes and modifications within the spirit and scope of the invention will become apparent to those skilled in the art from the detailed description.

### BRIEF DESCRIPTION OF THE DRAWING

FIG. 1. Lentiviral vector packaging plasmid test in the HEK293-6E cell line at various plasmid ratios.

FIG. 2. Lentiviral vector (LVV) production plasmid ratio test. A. Plasmid ratio test comparing a packaging system comprising a simple gene of interest (GOI) to a complex gene of interest (GOI). The key provided in the figure identifies the host cell line as 293-6E (HEK293-6E), VPC, or Expi293F. Plasmid ratios are mass ratios provided in the following form: Gag/pol plasmid: Rev plasmid: Envelope plasmid (VSV-G): Transfer plasmid (GOI). Plasmid ratio A is 2:1.75:1:20; plasmid ratio B is 4:4:1:8; and plasmid ratio C is 1.1:1:1.1:3.2. B. LVV functional titers were determined using the HEK293T or Jurkat cell lines to show relative measurements of the titers depending on the cell lines used. The concentrated LVV stocks incorporating a simple gene of interest (GOI) or a complex GOI were tested for their functional titers.

FIG. 3. Lentiviral vector (LVV) production method test. A. An LVV production method test was performed using the 293-6E (HEK293-6E) and the VPC cell lines harboring either a simple gene of interest (GOI) or a complex GOI. For the small and simple GOI, a standard plasmid expressing enhanced green fluorescent protein (eGFP) was used (pALD-Lenti-eGFP from Aldevron, Madison WI), resulting in a heterologous insert size of about 2 kb. The exemplary large and complex GOI consisted of multiple expressed genes (including eGFP used for titer readout) and had a total insert size of about 5 kb. The histogram reveals the functional titer of LVV in transducing units per mL (TU/mL) as a function of the type of GOI, i.e., simple GOI or complex GOI. The key provided in the figure identifies the host cell as either 293-6E or VPC and identifies the production method as either Method A or Method B supplemented with the Opti-Plex complexation solution. Method A is as described in Examples 1 and 2. Method B is as described in Example 3 and in U.S. Pat. Pub. No. 2018/0135077A1, incorporated herein by reference in relevant part. The Opti-Plex complexation solution is described in Example 3. B. The LVV production method was tested with a large and complex gene of interest (GOI) in the VPC cell line. The histogram shows the functional titer of LVV in transducing units per mL (TU/mL) for each of the three tested conditions, as revealed in the key (VPC cell line subjected to production Method A, production Method B supplemented with the Opti-Plex complexation solution, or production Method B supplemented with the Opti-MEM complexation solution, as described in Example 3).

FIG. 4. The production of LVV comprising a large and complex gene of interest (GOI) was assessed. A. LVV production in the VPC cell line was assessed by functional titer measured as the concentration of transducing units per mL (TU/mL). The results are presented graphically showing functional titer as a function of total plasmid concentration in μg/mL. Cells were harvested for assessment of functional titers on either day 2 or day 3, as indicated in the key. Plasmid ratios B and C are as defined in the brief description of FIG. 2. B. LVV production in the VPC cell line was assessed by p24 physical titer measured in lentiviral particles per mL (LP/mL). The p24 viral capsid protein was measured using ELISA, as described in Example 4. Cells were harvested for assessment of p24 concentration on either day 2 or day 3, as indicated in the key. Plasmid mass ratios B and C are as defined in the brief description of FIG. 2. C. LVV production in the VPC cell line was assessed by the percent specific activity of the LVV containing the complex GOI. Percent specific activity was a measure of the transducing units per lentiviral particle (% TU/LP). Cells were harvested for determinations of percent specific activity on either day 2 or day 3, as indicated in the key. Plasmid mass ratios B and C are as defined in the brief description of FIG. 2.

FIG. 5. The effect of a transfection complexation solution on production of LVV comprising a large and complex gene of interest (GOI) was determined. A. LVV production in the VPC cell line was assessed by functional titer measured as the concentration of transducing units per mL (TU/mL). Results are presented in a histogram showing the functional titer achieved with plasmid mass ratio B or plasmid mass ratio C. For each plasmid ratio, LVV transfection was performed in either the FreeStyle transfection complexation solution or the LV-MAX transfection complexation solution. Plasmid mass ratios B and C are as defined in the brief description of FIG. 2. B. LVV production in the VPC cell line was assessed by measuring the p24 capsid protein titer (LP/mL). Results are presented in a histogram showing the p24 titer achieved with plasmid mass ratio B or plasmid mass ratio C. For each plasmid mass ratio, LVV transfection was performed in either the FreeStyle transfection complexation solution or the LV-MAX transfection complexation solution. Plasmid mass ratios B and C are as define in the brief description of FIG. 2. C. LVV production in the VPC cell line was assessed by determining the percent specific activity of LVV comprising a complex GOI. Results are presented in a histogram showing the percent specific activity of LVV in transducing units per lentiviral particle (TU/LP). Percent specific activities were determined for production using plasmid mass ratio B or plasmid mass ratio C. For each plasmid ratio, LVV transfection was performed in either the FreeStyle transfection complexation solution or the LV-MAX transfection complexation solution. Plasmid mass ratios B and C are as defined in the brief description of FIG. 2.

FIG. 6. The effect of altering the relative mass ratio of the amount of transfection reagent to the total amount of plasmids on production of LVV comprising a large and complex gene of interest (GOI) was assessed by measuring functional titer (TU/mL) of the produced LVV. Results are presented as a histogram showing the functional titer (TU/mL) of produced LVV for production in varying amounts of transfection reagent relative to total plasmid amount. The relative amounts are presented as mass ratios of total plasmids (μg) to transfection reagent (μg), such as the PEI-MAX or PEIpro transfection reagent.

FIG. 7. LVV production increase resulting from improvement to the LVV production method. A. Production of LVV comprising a large and complex gene of interest (GOI) was measured by functional titer in transducing units per mL (TU/mL). The histogram shows the production yields from the VPC cell line measured by functional titers in transducing units per mL (TU/mL) for LVV comprising the complex GOI using either the original Method A or an improved variant of Method A. Original Method A used plasmid mass ratio A at a total plasmid concentration of 2.5 μg/mL with the day 3 harvest (see FIG. 2 and the brief description thereof) and improved Method A showed the day 2 harvest using plasmid mass ratio C at a total plasmid concentration of 1.5 μg/mL (see FIG. 4A and the brief description of FIG. 2). B. Production of LVV comprising a large and complex GOI was measured by p24 capsid protein titer also known as p24 physical titer in lentiviral particles per mL (LP/mL). The LVVs described in FIG. 7A were used to measure p24 capsid titer using ELISA (see Example 4). C. Production of LVV comprising a large and complex GOI was assessed by determining the percent specific activity (see FIG. 4C). Percent specific activity was a measure of the transducing units per lentiviral particle (% TU/LP) and the data displayed in FIGS. 7A and 7B were used.

### DETAILED DESCRIPTION

The disclosure features an animal-free composition and method for lentiviral vector production comprising a) culturing eukaryotic producer cells in animal-free media, b) providing codon-optimized lentiviral packaging plasmids, c) packaging transgene expression cassettes of varying sizes and complexity, d) optimizing transfection conditions, and e) enhancing lentiviral production with sodium butyrate. The animal-free composition and methods contain no human or animal-derived protein (e.g., no serum, no transferrin) present in the media or reagents used to manufacture the lentiviral vector. The disclosed methods use an HEK293-based suspension cell line for production (not the “HEK293T” cell line used in virus titer assays disclosed herein) that is optimized to grow in chemically defined (animal-free) medium. Thus, the cells used for LVV production are never exposed to medium or other components containing an animal-derived substance, such as is found in bovine serum albumin (BSA) and FBS.

Unless specifically noted in the following Examples, the materials and methods used in the experimental work described in those Examples are presented here. Lentiviral vectors (LVV) were produced by transient transfection of HEK293 cells adapted for suspension culture, such as Viral Production Cells (VPC; ThermoFisher Scientific), that were grown in LV-MAX Production Medium (ThermoFisher Scientific). The day before transfection, cells were seeded at about 2.5-3.5×10⁶ viable cells (vc)/mL in shake flasks at the desired scale and incubated at 37° C., 5% CO₂ with appropriate agitation per vessel used. Appropriate agitation per vessel used, as recited herein, means sufficient agitation, typically using a shaking or rotating platform or incubator, to prevent loss of culture medium, harm to the cells, or foaming while ensuring adequate aeration of the cells maintained in suspension culture, in order to support optimal cell growth. The next day, a four-plasmid transfection mix was prepared containing pALD-GagPol-K, pALD-Rev-K, pALD-VSV-G-K (Aldevron) and the appropriate transgene plasmid at a mass ratio of 1.1:1:1.1:3.2 (i.e., plasmid ratio C), respectively, in LV-MAX media (ThermoFisher Scientific) at 1.5 mg/L (1.5 mg of total plasmids per liter of LVV production volume) in 1/10 the volume of the total final production scale. As used herein, a “plasmid ratio” refers to a plasmid mass ratio unless otherwise expressly indicated or apparent from context. The plasmids were mixed with PEI-MAX (1.0 mg/mL at pH 7.0, Polyethylenimine Hydrochloride from Polysciences Inc.) at a mass ratio of 1:3 (total plasmids: PEI-MAX) and incubated for 10-15 minutes at room temperature. Alternatively, PEIpro (Polyplus Transfection) rather than PEI-MAX can be used as a transfection reagent. When using PEIpro, the same procedure applies as when using PEI-MAX, except the mass ratio of total plasmids to PEIpro is reduced to a ratio of 1:(1.5-2.0) (total plasmids: PEIpro). The transfection mix was added to the cultured cells at 4.0×10⁶ vc/mL, and cells were returned to the incubator. The next day, sodium butyrate was added to a concentration of 10 mM. The cell culture supernatant containing the lentiviral vector (LVV) was harvested 2 days post-transfection, clarified by centrifugation at 500×g for 10 minutes, and filtered through a 0.22 micron low-protein binding filter membrane. Harvest samples were collected by mixing the filtered LVV with 70% sucrose at a 9:1 ratio and stored at −80° C. When concentration of the LVV was desired, the harvested LVV was concentrated using high-speed centrifugation at about 12,000×g for 5-6 hours at 4-8° C., resuspended in about 1/500 of the starting volume in final formulation sucrose-phosphate-glutamate (SPG) buffer (218 mM Sucrose, 3.8 mM Potassium Phosphate Monobasic, 7.2 mM Potassium Phosphate Dibasic, 4.9 mM Potassium Glutamate) and stored at −80° C.

LVV infectious titers were determined by transducing either HEK293T or Jurkat cell lines with a series of serially diluted LVV stocks. LVV functional titers are commonly measured by assaying transgene expression in highly transducible cell lines, such as HEK293, HEK293T, and HT1080, though it is well known in the field that such titer values are typically higher than those obtained in clinically relevant target cells that have more limited transducibility. In order to obtain more clinically meaningful LVV titers, LVV functional titers were measured in a T cell-derived Jurkat cell line. In our experience, and as shown in FIG. 2B, LVV titers measured in the Jurkat cell line are several-fold lower compared to the titers obtained in the HEK293T cell line. Three days post-transduction, expression of transgene was measured by eGFP using flow cytometry (% positive cells), and the % positive cell values were converted into functional titers.

LVV physical titers were determined by quantification of the p24 viral capsid protein using p24 enzyme-linked immunosorbent assay (ELISA) using a commercially available kit (Alliance HIV-1 p24 Antigen ELISA Kit, PerkinElmer). In this ELISA, an antibody specific for HIV-1 p24 is coated on the assay plate to capture the p24 present in the sample. a second biotinylated antibody against HIV-1 p24 is added, and streptavidin conjugated to horseradish peroxidase (HRP) followed by ortho-phenylenediamine-HCl (OPD) substrate is added to determine the p24 concentration using a standard curve.

### EXAMPLES

### Example 1

### Comparison Study of Two Sets of LVV Packaging Plasmids

The HEK293 suspension cell line 293-6E from the National Research Council Canada, was utilized in producing lentiviral vectors (LVV). Cells were cultured in serum-free FreeStyle™ 293 Expression Medium (FreeStyle) from ThermoFisher Scientific, supplemented with Pluronic™ F-68 Non-ionic Surfactant (100×) at a working concentration of 0.1% using disposable sterile shake flasks at the desired scale and incubated at 37° C., 5% CO₂ with agitation sufficient to aerate cells retained in suspension without significantly damaging those cells.

Lentiviral vectors (LVVs) were produced by a serum-free process in 293-6E cells by transient transfection using two different LVV packaging plasmid sets. The Packaging Plasmid 1 set consisted of pMDO-Lgpr (GagPol) (SEQ ID NO:1), pRSV-Rev (Rev) (SEQ ID NO: 4), and pCIGO-Vg (VSV-G) (SEQ ID NO:7). The Packaging Plasmid 2 set contains optimized packaging constructs pALD-GagPol, pALD-Rev, and pALD-VSV-G, which are commercially available from Aldevron under the pALD-Lenti System. For the transgene, also known as the gene of interest (GOI), i.e., the exogenous nucleic acid, the pLENTI-EGFP construct from Aldevron was used with both packaging plasmid sets.

The day before transfection, cells were seeded at about 0.7×10⁶-0.8×10⁶ viable cells (vc)/mL. On the day of transfection, a four-plasmid mix containing GagPol, Rev, VSV-G and GOI was prepared at three different mass ratios of GagPol: Rev: VSV-G: GOI, i.e., ratio A (2:1.75:1:20), ratio B (4:4:1:8) and ratio C (1.1:1:1.1:3.2) at 0.5 mg/L (0.5 mg of total plasmids per liter of LVV production medium). The four-plasmid mix was prepared in FreeStyle™ 293 Expression Medium in 1/10 the volume of the total production scale. The four-plasmid mix in FreeStyle™ 293 Expression Medium was combined with the transfection reagent PEI MAX™ (1.0 mg/mL at pH 7.0, Polyethylenimine Hydrochloride from Polysciences Inc.) at a mass ratio of 1:3 (total plasmids: PEI-MAX). An exemplary LVV production scale is the 1 L scale LVV production in which the 1 L refers the final total volume after cells and transfection mix are combined. For the 1 L scale LVV production, 90% of the 1 L (i.e., 0.9L) comes from the cells and 10% of the 1 L (i.e., 0.1 L) comes from the transfection mix (plasmids mixed in complexation solution such as FreeStyle 293 expression medium+transfection reagent such as PEI-MAX). The four-plasmid mix with PEI-MAX™ was incubated for about 10-15 minutes at room temperature to form the transfection mixture. The transfection mixture was added to the prepared cells grown to about 1.0×10⁶-2.0×10⁶ viable cells (vc)/mL, and the cells with the transfection mixture were returned to the incubator. The next day, sodium butyrate was added to a concentration of 10 mM. The cell culture supernatant containing LVVs was harvested three days post-transfection, clarified by centrifugation at 500×g for 10 minutes, and filtered through a 0.22 micron low-protein-binding filter membrane. Harvested samples were collected by mixing the filtered LVV with 70% sucrose at a 9:1 ratio (harvested LVV: 70% sucrose), corresponding to a working concentration of 7% sucrose, and stored at −80° C.

Lentiviral vector (LVV) infectious titers (also known as functional titers) were determined by transducing the HEK293T cell line with a series of serially diluted LVV stocks. Three days post-transduction, enhanced Green Fluorescent Protein (eGFP) transgene expression was measured by flow cytometry (% positive cells), and the % positive cells were converted into the functional titers.

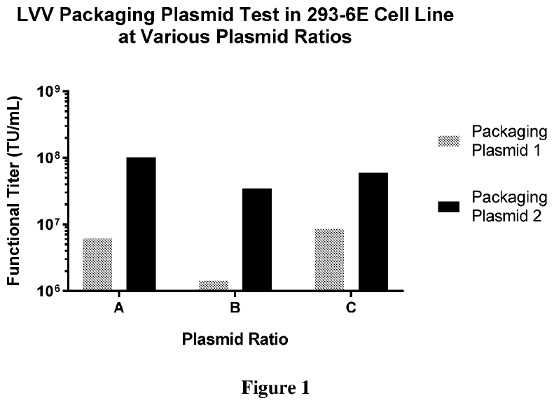

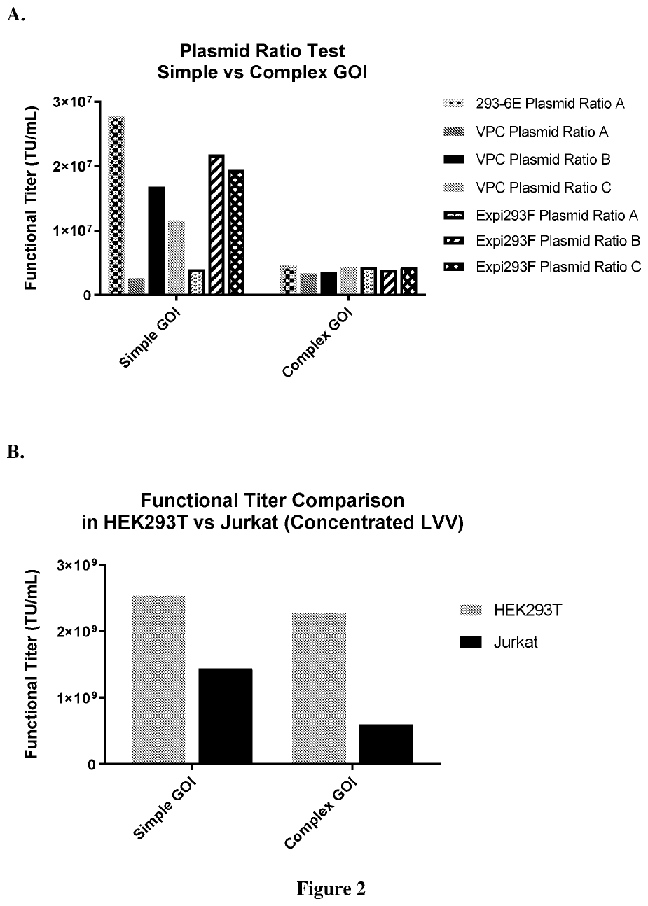

FIG. 1 shows the LVV production yields measured by functional titers for the production runs performed using two sets of LVV packaging plasmids at three different plasmid ratios. LVV production yields increased by more than one log using the Packaging Plasmid 2 set, which consists of the improved LVV packaging plasmid set from Aldevron. Plasmid Ratio A resulted in the highest functional titer. These results showed superior virus titers being obtained when using codon-optimized packaging plasmids together with optimized plasmid ratios.

### Example 2

### Impact of Transgene Size/Complexity and Plasmid Ratio on LVV Production Yields

Three different HEK293 suspension cell lines were compared for production of LVV under serum-free conditions. The first cell line was the 293-6E cell line from the National Research Council Canada, which was cultured as described in Example 1. The second cell line was Gibco™ Viral Production Cells (VPC) from ThermoFisher Scientific, which was cultured in serum-free LV-MAX™ Production Medium (LV-MAX) from ThermoFisher Scientific. The third cell line was Expi293F™ Cells (Expi293F) from ThermoFisher Scientific, which was cultured in serum-free Expi293™ Expression Medium (ThermoFisher Scientific). All three cell lines were cultured in disposable sterile shake flasks at the desired scale and incubated at 37° C., 5% CO₂ with agitation sufficient to aerate the cells retained in suspension without significantly damaging those cells.

LVVs were produced in the above-mentioned cell lines by transient transfection under serum-free conditions. The day before transfection, cells were seeded at about 0.7×10⁶-0.8×10⁶ vc/mL for the 293-6E cell line and about 2.5×10⁶-3.5×10⁶ vc/mL for the VPC and Expi293F cell lines. On the day of transfection, a four-plasmid mix containing pALD-GagPol, pALD-Rev, pALD-VSV-G and the appropriate GOI was prepared at three different mass ratios, i.e., A (GagPol: Rev: VSV-G: GOI=2:1.75:1:20), B (4:4:1:8) and C (1.1:1:1.1:3.2). The relative quantities of plasmids tested in these experiments are provided in terms of mass ratios and molar ratios in Table 1. For all three plasmid ratios, a total plasmid concentration of 2.5 mg/L (2.5 mg of total plasmids per 1 L of LVV production volume) was used for VPC and Expi293F cell lines according to the manufacturer's instruction. The 293-6E cell line was included as a positive control with the previously optimized plasmid ratio A condition at 0.5 mg/L total plasmid. For the simple GOI condition, a pLENTI-EGFP construct (Aldevron; about 2 kb total insert size for the heterologous nucleic acid) expressing enhanced green fluorescent protein (eGFP) was used, and for the large and complex GOI, a TCR-EGFP multigene construct (about 5 kb total insert size) co-expressing a T cell receptor (TCR) and eGFP was used. The transfection mixture was prepared as described in Example 1 and added to the appropriate cultured cells at about 1.0×10⁶-2.0×10⁶ vc/mL for 293-6E cells and about 4.0×10⁶ vc/mL for VPC and Expi293F cells. The rest of the LVV production process was carried out as described in Example 1.

TABLE 1

| | | | “Molar Plasmid | “Molar Plasmid |
|---|---|---|---|---|
| Plasmid Ratio | | “Mass” Plasmid | Ratio (Simple | Ratio (Complex |
| Origin | Plasmid Type | Ratio (Any GOI) | eGFP GOI) | TCR-eGFP GOI) |
| Ratio A | Gag/Pol:Rev:VSVG:GOI | 2:1.75:1:20 | 1.0:2.4:1.1:13.7 | 1.0:2.4:1.1:9.6 |
| Ratio B | Gag/Pol:Rev:VSVG:GOI | 4:4:1:8 | 1.9:5.2:1.0:5.1 | 1.9:5.2:1.0:3.6 |
| Ratio C | Gag/Pol:Rev:VSVG:GOI | 1.1:1:1.1:3.2 | 1.0:2.6:2.1:4.0 | 1.0:2.6:2.1:2.8 |
| | | | “Molar Plasmid | “Molar Plasmid |
| Plasmid Ratio | | “Mass” Plasmid | Ratio (Simple | Ratio (Complex |
| Origin¹ | Plasmid Type | Ratio (Any GOI) | eGFP GOI) | TCR-eGFP GOI) |
| Ratio A | Gag/Pol:Rev:VSVG:GOI | 0.08:0.07:0.04:0.81 | 0.05:0.13:0.06:0.75 | 0.07:0.17:0.08:0.68 |
| Ratio B | Gag/Pol:Rev:VSVG:GOI | 0.24:0.24:0.06:0.47 | 0.14:0.40:0.08:0.39 | 0.16:0.45:0.09:0.31 |
| Ratio C | Gag/Pol:Rev:VSVG:GOI | 0.17:0.16:0.17:0.50 | 0.10:0.27:0.22:0.41 | 0.12:0.31:0.25:0.33 |

¹Lower panel - plasmid masses normalized to a total plasmid mass of 1

The LVV functional titers were determined by the method described in Example 1, except that HEK293T target cells were substituted with a more clinically meaningful T-cell target, i.e., the Jurkat cell line. In our experience, LVV titers measured in the Jurkat cell line are typically several-fold lower compared to titers obtained in the HEK293T cell line.

FIG. 2A shows the LVV production yields measured by functional titers comparing the productions achieved with simple GOI versus complex GOI in three different cell lines, i.e., 293-6E, VPC and Expi293F. For the VPC and Expi293F cell lines, data from three different plasmid ratios are shown. For the small heterologous nucleic acid or single transgene cassette of about 2 kb (simple GOI), functional LVV titers were highest from the 293-6E cell line, while titers generated in VPC and Expi293F cells varied depending on the plasmid ratio. As expected, LVV production yields from all three cell lines decreased significantly when using a large transgene cassette (about 5 kb insert size), which also expressed two transgenes (complex GOI). However, for the large and complex GOI, there were no significant differences observed in the functional titers when varying the plasmid ratios. Therefore, production of LVV carrying large and complex transgene cassettes is suboptimal and cannot simply be improved by changing the plasmid ratios used for transfection.

FIG. 2B shows LVV functional titers determined using the HEK293T or Jurkat cell lines to show relative measurements of the titers depending on the cell lines used. For this study, the concentrated LVV stocks prepared using the concentration method described

herein (see paragraph [0028]) were utilized. The concentrated LVV stocks incorporating a small and simple gene of interest (GOI) or a large and complex GOI as heterologous nucleic acids were tested for their functional titers. In both cases, LVV titers measured in the Jurkat cell line were several-fold lower compared to the titers obtained in the HEK293T cell line.

### Example 3

### Comparison Study of Two Optimized LVV Production Methods

LVVs were produced in the 293-6E and VPC cell lines by transient transfection using either the LVV production method we developed (“Method A”) or a commercially marketed LVV production method by ThermoFisher (“Method B”). Method A involved the serum-and animal-free transfection step using PEI-MAX transfection reagent and chemically defined medium, FreeStyle for the 293-6E cell line, or LV-MAX for the VPC cell line, at a total concentration of 0.5 μg/mL for the 293-6E cell line, or 2.5 μg/mL for the VPC cell line as described in Examples 1 and 2. Method A also involved a simple enhancer step of adding sodium butyrate to a 10 mM concentration the day after transfection for both the 293-6E and VPC cell lines, as described in Examples 1 and 2. For Method B, the LV-MAX Transfection Kit from ThermoFisher Scientific was used, which includes an optimized set of complex and proprietary reagents, LV-MAX Supplement, LV-MAX Transfection Reagent, and LV-MAX Enhancer. The transfection kit is based on an optimized procedure previously described in U.S. Pat. Pub. No. 2018/0135077A1, incorporated herein by reference in relevant part, which uses a culture supplement containing a complex mix of nutrients, a production enhancer containing sodium propionate, sodium butyrate, caffeine, and a lipofection reagent containing DHDMS, DOPE, and cholesterol. For Method B, LVV production was carried out using the included culture supplement, transfection enhancer, and transfection reagent together with either animal-free Opti-Plex™ or xeno-free, but human-derived Opti-MEM™ transfection complexation solutions, according to the manufacturer's instruction. Briefly describing Method B, on the day of transfection, a culture supplement was added at 5% of total LVV production scale to the prepared cells. For Method B transfection, the plasmid mixture and LV-MAX Transfection Reagent were pre-diluted in either animal-free Opti-Plex™ or xeno-free, but human-derived, Opti-MEM™ transfection complexation solutions, and incubated for one minute. The diluted plasmids and LV-MAX Transfection Reagent were then mixed, and incubated for 10 minutes at room temperature. The transfection mixture was added to the prepared cells with LV-MAX Supplement. For LVV production Method B in the 293-6E cell line, Packaging Plasmid 2 with Plasmid Ratio A at a total concentration of 0.5 μg/mL was used, as described in Example 1. For LVV production Method B in the VPC cell line, the Packaging Plasmid 2 with Plasmid Ratio B at a total concentration of 2.5 μg/mL was used, as described in Example 2. For Method B, LV-MAX Enhancer was added at 4% of total LVV production scale the day after transfection, for both 293-6E and VPC cell lines. Consistent with the foregoing description of Methods A and B, Method A can be characterized as a PEI-based transfection method with the simple addition of an enhancer step by adding sodium butyrate, as described in Examples 1 and 2. In general terms, the comparison of Method A and Method B is a comparison of a PEI-MAX+sodium butyrate production method (Method A) and the commercial production method using ThermoFisher s CT transfection kit (Method B), which includes a lipid-based transfection reagent, a supplement, and a complex enhancer mixture, as noted above.

Cell culture supernatants containing LVVs were harvested at day 2 and day 3 post-transfection, as indicated above, and processed as described in Examples 1 and 2. The LVV functional titers were determined as described in Example 2.

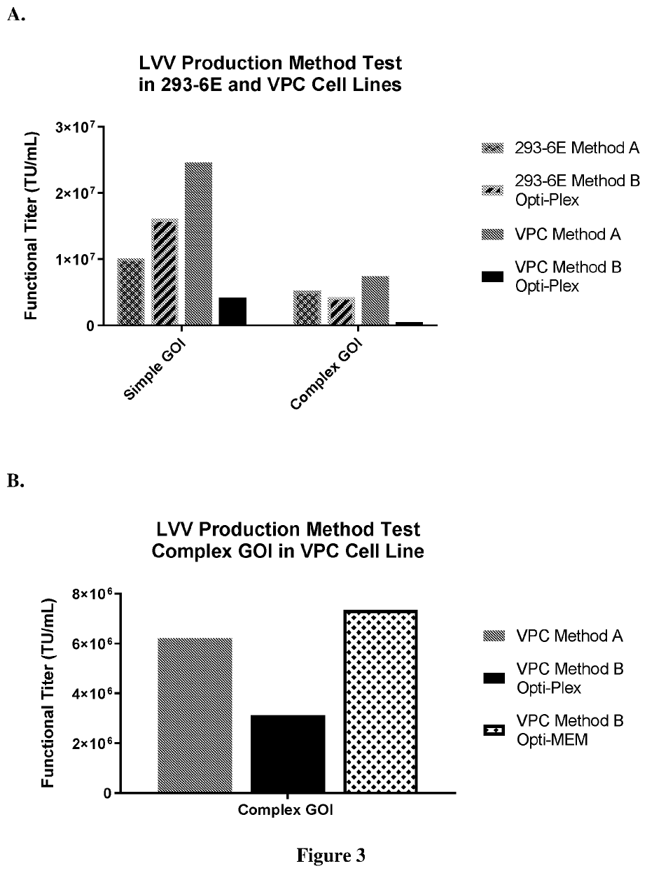

FIG. 3A shows the LVV production yields measured by functional titers comparing the productions achieved using the LVV production Method A (proprietary method described herein) versus Method B (LV-MAX Transfection Kit from ThermoFisher Scientific) in the 293-6E and VPC cell lines, with production obtained using a small and simple GOI compared to production achieved using a large and complex GOI. For each cell line, the conditions with the overall highest functional titers are displayed, which were the day 3 harvest data for the 293-6E cell line, and the day 2 harvest data for the VPC cell line. For the 293-6E cell line, Method B showed modest improvement in production yields compared to Method A with the simple GOI, but not with the complex GOI. For the VPC cell line, Method A resulted in markedly higher production yields than Method B for both simple and complex GOIs.

FIG. 3B shows the LVV production yields measured by functional titers comparing the productions achieved using LVV production Method A versus Method B in the VPC cell line with complex GOI. For Method B, the animal-free Opti-Plex™ complexation solution (Opti-Plex) was compared to the xeno-free, but human-derived, Opti-MEM™ (Opti-MEM) complexation solution. For LVV production achieved using a completely serum-and animal-free process, Method B in combination with Opti-Plex resulted in significantly lower LVV production yield than was achieved with Method A. For Method B, the production yield improved significantly only when animal-derived Opti-MEM was introduced into the process. Together, these results show that when packaging a large and complex transgene cassette, the complex mix of reagents used by Method B does not result in superior virus yields compared to Method A, which uses simple and cost-effective reagents (PEI and sodium butyrate), when an animal-free production process is utilized. LVV production Method B utilizes Opti-MEM (see U.S. Pat. Pub. No. 2018/0135077A1), which is serum-free but contains human-derived transferrin, which adds a potential safety risk in clinical production settings because of the inclusion of animal protein (e.g., transferrin) in the production method.

### Example 4

### Impact of Total Plasmid Concentration and Harvest Time on LVV Production Yield

LVVs were produced in the VPC cell line by transient transfection with plasmids comprising the large and complex GOI using the plasmid ratios B and C and following the transfection process described in Example 2. For this Example, a range of total plasmid concentrations from 0.5 to 2.5 μg/mL were tested. The cell culture supernatant containing LVVs was harvested at day 2 and at day 3 post-transfection and processed as described in Example 2. LVV functional titers were determined as described in Example 2.

For select LVV samples, LVV physical titers were determined by quantification of the p24 viral capsid protein using a p24 enzyme-linked immunosorbent assay (ELISA) with a commercially available kit (Alliance HIV-1 p24 Antigen ELISA Kit, PerkinElmer). In this ELISA, an antibody specific for HIV-1 p24 is coated on the assay plate to capture the p24 present in the sample, a second biotinylated antibody against HIV-1 p24 is added, and streptavidin conjugated to horseradish peroxidase (HRP) followed by ortho-phenylenediamine-HCl (OPD) substrate is added to determine the p24 concentration colorimetrically using a standard curve.

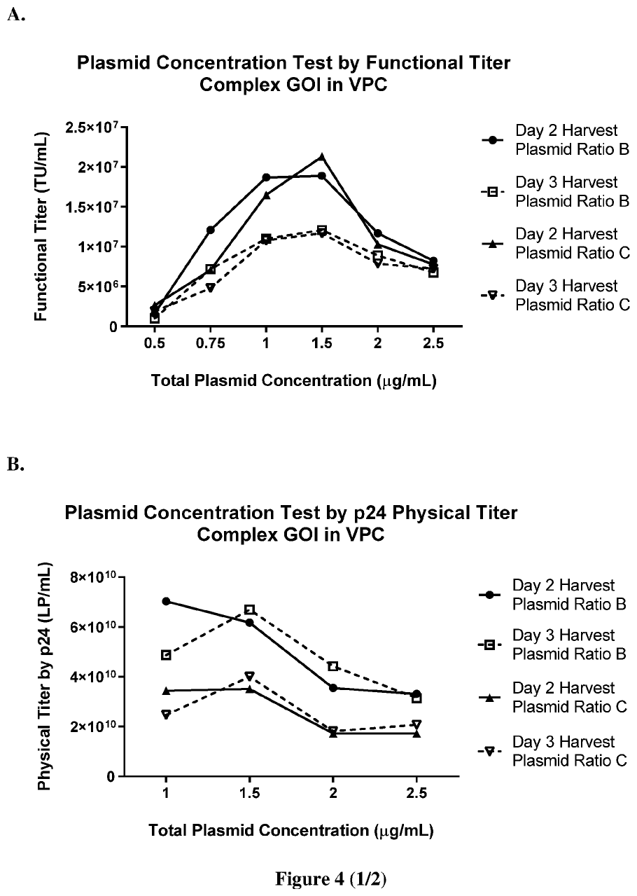

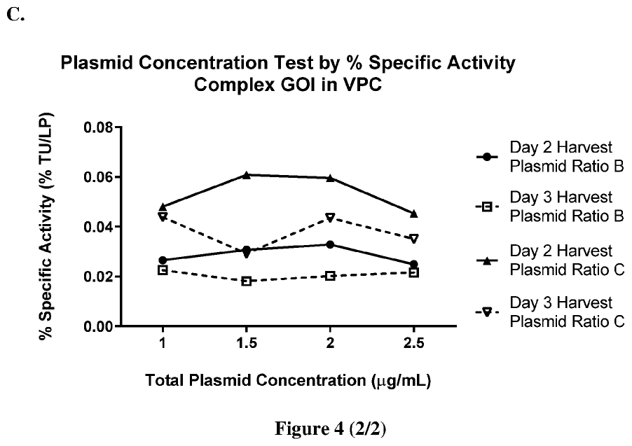

FIG. 4A shows the LVV production yields measured as functional titers for various total plasmid concentrations used during the transfection step. The data from total plasmid concentrations ranging from 0.5 to 2.5 μg/mL, using plasmid ratios B and C, and for day 2 and day 3 harvests, are shown. Plasmid ratios B and C resulted in comparable functional titers, with both reaching the highest titers at around 1 to 1.5 μg/mL total plasmid. For both plasmid ratios B and C, significantly higher functional titers were observed in the day 2 harvest than in the day 3 harvest.

FIG. 4B shows the corresponding LVV capsid yields measured by p24 physical titers for a subset of total plasmid concentrations used during the transfection step. The data from a range of 1 to 2.5 μg/mL total plasmid, using plasmid ratios B and C, and for day 2 and day 3 harvests are shown. A total plasmid concentration of 1 to 1.5 μg/mL resulted in the highest p24 capsid titers, though no significant difference in capsid titers was observed between the day 2 and day 3 harvests. Across the range of total plasmid concentrations studied, LVV productions performed with plasmid ratio B resulted in significantly higher p24 capsid titers than those that used plasmid ratio C.

FIG. 4C shows the specific activity (SA) of LVV produced across a range of total plasmid concentrations and different plasmid ratios and harvest days. The specific activity measures the percentage of functional LVV particles (Transducing Units, TU) that are capable of transducing cell lines among the total number of viral capsids (Lentiviral Particles, LP). The % SA is calculated by dividing the functional titer by the p24 physical titer and multiplying by 100. LVV productions using plasmid ratio C resulted in significantly higher SA compared to plasmid ratio B across the tested range of total plasmid concentrations. For both plasmid ratios, day 2 harvests resulted in higher % specific activities than day 3 harvests. LVV productions with plasmid ratio C at around 1.5 to 2.0 μg/mL of total plasmid resulted in the highest % specific activities.

Together, these results show that functional titer yields and specific activity of LVV carrying large and complex transgene cassettes can be significantly improved by optimized combinations of plasmid ratio and total plasmid concentration transfection parameters, combined with the optimal harvest day.

### Example 5

### Transfection Complexation Solution Test for VPC Cell Line

LVVs were produced in the VPC cell line by transient transfection with the large and complex GOI using plasmid ratios B and C and following the transfection process described in Example 2. For this Example, the four-plasmid mix was prepared in either FreeStyle™ or LV-MAX at 1.5 μg/mL plasmid concentration. The cell culture supernatant containing LVVs was harvested at day 2 post-transfection and processed as described in Example 2.

LVV functional titers were determined as described in Example 2, and LVV physical titers were determined as described in Example 4.

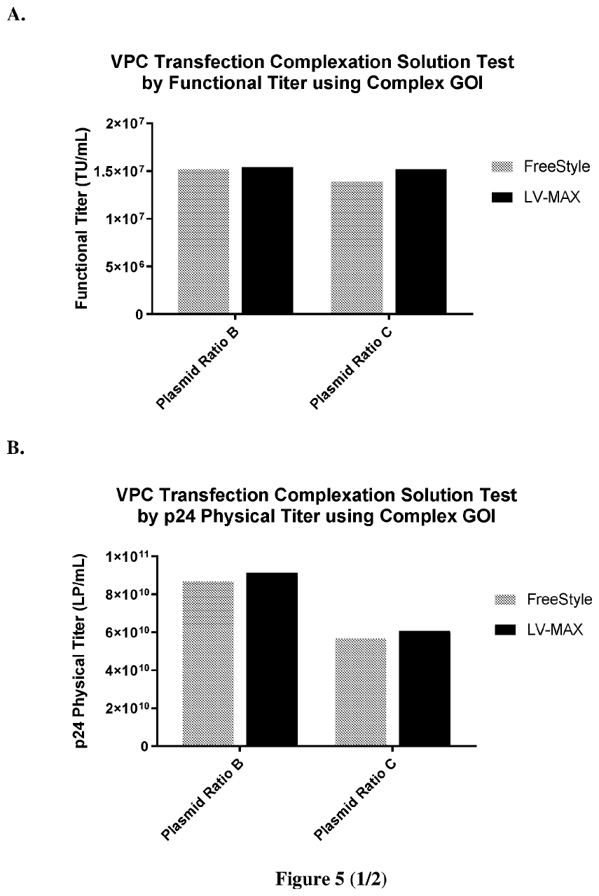

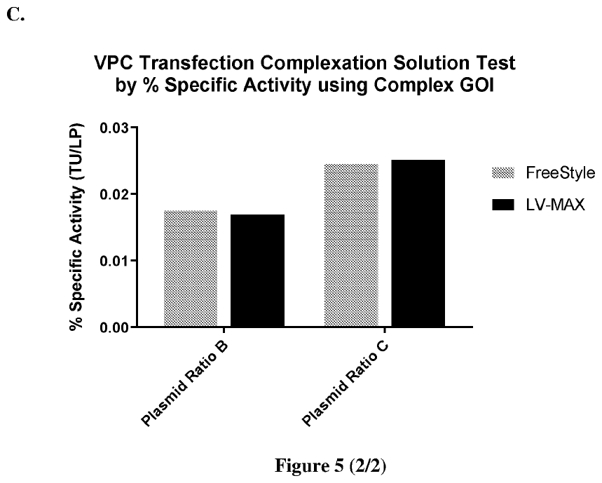

FIG. 5A shows the LVV production yields measured as functional titers for two different transfection complexation solutions, i.e., FreeStyle and LV-MAX, using plasmid ratios B and C. The data from FreeStyle and LV-MAX resulted in comparable functional titers for both plasmid ratios B and C.

FIG. 5B shows the corresponding LVV capsid yields measured by p24 physical titers for the samples listed in FIG. 5A. The data from FreeStyle and LV-MAX resulted in comparable p24 capsid titers for both plasmid ratios B and C. LVV production levels achieved with plasmid ratio B, however, resulted in significantly higher p24 capsid titers than those that used plasmid ratio C.

FIG. 5C shows the specific activity (SA) of LVV produced using two different transfection complexation solutions using different plasmid ratios. The SA is calculated as described in FIG. 4C of Example 4. LVV productions performed using FreeStyle and LV-MAX resulted in comparable SAs for both plasmid ratios B and C. LVV production levels achieved with plasmid ratio C, however, resulted in a significantly higher SA compared to the SA for plasmid ratio B across all conditions.

Together, these results show that comparable LVV production yields were achieved from two different transfection complexation solutions, i.e., FreeStyle and LV-MAX. This enables the LVV production process to be more streamlined by using the same LV-MAX throughout the process. The results also confirm some of the findings described in the figures cited in Example 4 that plasmid ratio C is better than plasmid ratio B for packaging the large and complex GOI, resulting in significantly higher SA than those that used plasmid ratio B.

### Example 6

### Comparison Study of PEI-Based Transfection Reagents

LVVs were produced in the VPC cell line by transient transfection using the process described in Example 2. For this Example, the large and complex GOI was produced under optimized conditions, i.e., plasmid ratio C and a 1.5 μg/mL total plasmid concentration in LV-MAX for transfection. A PEI MAX™-based transfection was set up as described in Example 2. For PEIpro® (Polyplus Transfection)-based transfection, plasmids and PEIpro were prepared according to manufacturer's instruction and at various mass ratios of plasmid to PEIpro, covering a range of 1:1 to 1:3.0 (total plasmids:PEIpro).

Cell culture supernatants containing LVV were harvested on day 2 and day 3 post-transfection and processed as described in Example 2. The LVV functional titers were determined as described in Example 2.

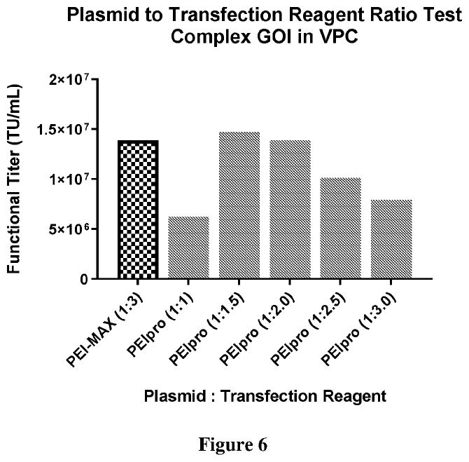

FIG. 6 shows the LVV production yields measured by functional titers when comparing PEI-based transfection reagents and the plasmid-to-transfection-reagent ratios tested for PEIpro. For PEIpro, the ratios of plasmid to transfection reagent between 1:1.5 to 1:2.0 resulted in the highest functional titers, and these titers were comparable to the data observed from the production achieved with an optimized ratio of PEI-MAX. These results show that high titers of LVV packaging a large and complex transgene cassette can be obtained when using optimized ratios of different PEI-based transfection reagents, such as PEI-MAX and PEIpro.

### Example 7

### Overall LVV Production Improvement Achieved

LVVs were produced in the VPC cell line with the large and complex GOI by transient transfection using PEI-MAX as described in Examples 2, 3 and 4. The displayed data herein were taken from the figures (FIG. 2A and FIG. 4A) included in previous Examples to illustrate the overall LVV production increase resulting from improvement to the LVV production method. The data labeled as Original Method A were taken from VPC Plasmid Ratio A in FIG. 2A, where LVVs were produced using plasmid ratio A at total plasmid concentration of 2.5 μg/mL with day 3 harvest as described in Example 2. The data labeled as Improved Method A were taken from Day 2 Harvest Plasmid Ratio C at 1.5 μg/mL total plasmid concentration in FIG. 4A as described in Example 4. The Method A is described in detail in Example 3. The LVV functional titers were determined as described in Example 2. The LVV p24 physical titers were determined as described in Example 4. The percent specific activity was calculated as described in Example 4.

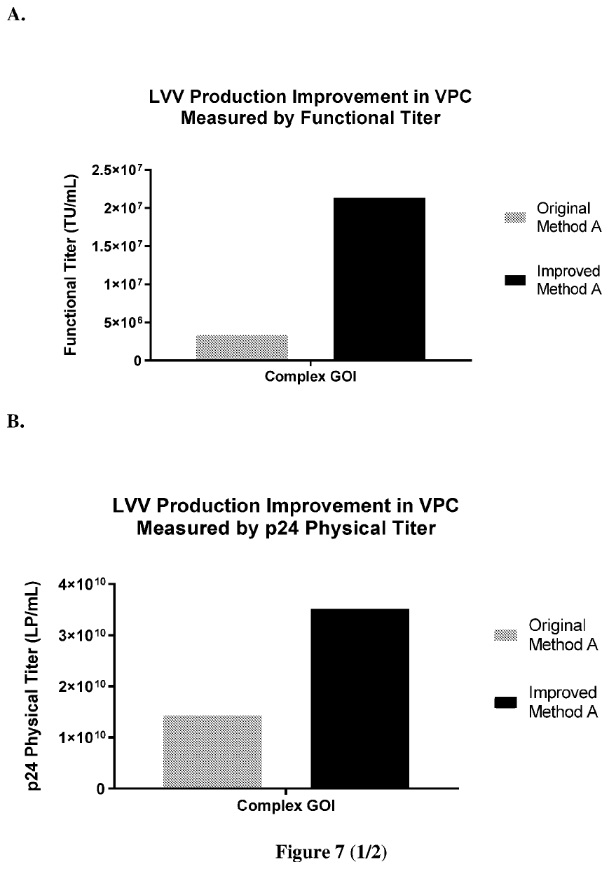

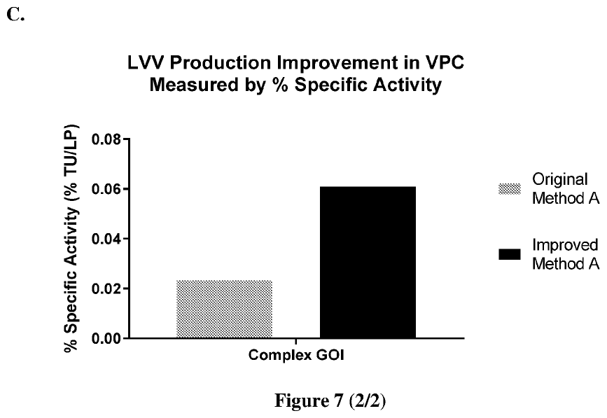

FIG. 7 shows LVV production increase resulting from improvement to the LVV production method, specifically Method A, in the VPC cell line. Production of LVV comprising the large and complex GOI was measured by functional titer in transducing units per mL (TU/mL). Original Method A used sub-optimal conditions (i.e., plasmid ratio A, total plasmid concentration of 2.5 μg/mL and day 3 harvest) and Improved Method A used optimized conditions (i.e., plasmid ratio C, total plasmid concentration of 1.5 μg/mL and day 2 harvest) as described above. The overall LVV production yields increased about 6.4-fold based on the functional titers, 2.5-fold based on the p24 physical titers, and about 2.6-fold based on the percent specific activities using the Improved Method A compared to production using the Original Method A.

The disclosed subject matter has been described with reference to various specific embodiments and techniques. It should be understood, however, that many variations and modifications may be made while remaining within the spirit and scope of the disclosed subject matter.

What is claimed is:

1. A lentiviral plasmid packaging composition comprising a gag/pol plasmid, a rev plasmid, an envelope plasmid, and a transfer plasmid, wherein the plasmids are present in a mass ratio of 1.1 gag/pol plasmid:1 rev plasmid:1.1 envelope plasmid:3.2 transfer plasmid, wherein the plasmid packaging composition lacks a wild-type lentiviral 5′ long terminal repeat (LTR) promoter, and wherein the transfer plasmid comprises a heterologous nucleic acid greater than 2 kb.

2. The lentiviral plasmid packaging composition of claim 1 wherein the envelope plasmid comprises a coding region for vesicular stomatitis virus G protein (VSV-G).

3. The lentiviral plasmid packaging composition of claim 1 wherein the heterologous nucleic acid is at least 4 kb.

4. The lentiviral plasmid packaging composition of claim 3 wherein the heterologous nucleic acid is at least 5 kb.

5. The lentiviral plasmid packaging composition of claim 3 wherein the heterologous nucleic acid is 4-8 kb.

6. The lentiviral plasmid packaging composition of claim 5 wherein the heterologous nucleic acid is 4-7.5 kb.

7. The lentiviral plasmid packaging composition of claim 5 wherein the heterologous nucleic acid is 4-5 kb.

8. The lentiviral plasmid packaging composition of claim 1 wherein the heterologous nucleic acid is greater than 8 kb.

9. The lentiviral plasmid packaging composition of claim 1 wherein the heterologous nucleic acid encodes a T-cell receptor, a chimeric antigen receptor, or a multi-gene complex.

10. The lentiviral plasmid packaging composition of claim 1 wherein the total DNA concentration of gag/pol plasmid, rev plasmid, envelope plasmid, and transfer plasmid is 0.25-2.5 μg/mL.

11. The lentiviral plasmid packaging composition of claim 10 wherein the total DNA concentration of gag/pol plasmid, rev plasmid, envelope plasmid, and transfer plasmid is 1.0-2.0 μg/mL.

12. The lentiviral plasmid packaging composition of claim 1 that lacks a lentiviral tat gene.

13. The lentiviral plasmid packaging composition of claim 1 further comprising a complexation solution.

14. The lentiviral plasmid packaging composition of claim 13 wherein the complexation solution is OPTI-MEM, Opti-plex, FreeStyle™, or LV-MAX.

15. The lentiviral plasmid packaging composition of claim 1 further comprising a transfection reagent.

16. The lentiviral plasmid packaging composition of claim 15 wherein the transfection reagent is PEIpro or PEI-MAX.

17. The lentiviral plasmid packaging composition of claim 16 wherein the transfection reagent is PEIpro.

18. The lentiviral plasmid packaging composition of claim 15 wherein the mass of total plasmid and the mass of transfection reagent are present in a ratio between 1 total plasmid mass:1.0 transfection reagent mass and 1 total plasmid mass:3.0 transfection reagent mass.

19. The lentiviral plasmid packaging composition of claim 1 wherein the gag/pol plasmid comprises the sequence set forth at SEQ ID NO: 1, the rev plasmid comprises the sequence set forth at SEQ ID NO:4, and the envelope plasmid comprises the sequence set forth at SEQ ID NO:7.

20. A method of producing a recombinant lentiviral vector comprising:
(a) preparing a mixture comprising the lentiviral plasmid packaging composition of claim 1 in complexation solution and a transfection reagent in a mass ratio of 1 total plasmid mass:greater than 1 transfection reagent mass;
(b) infecting a eukaryotic host cell with the mixture; and
(c) culturing the infected host cell, thereby producing the recombinant lentiviral vector.

21. The method of claim 20 wherein the mixture comprising the lentiviral plasmid packaging composition of claim 1 in complexation solution and the transfection reagent is incubated for at least 10 minutes before infecting the eukaryotic host cell.

22. The method of claim 21 further comprising adding sodium butyrate to the infected host cell culture about one day after infection.

23. The method of claim 22 wherein about 10 mM sodium butyrate is added to the infected host cell culture.

24. The method of claim 20 wherein the mixture comprises a mass ratio of 1 total plasmid mass:3 transfection reagent mass.

25. The method of claim 20 wherein the envelope plasmid comprises a coding region for vesicular stomatitis virus G protein (VSV-G).

26. The method of claim 20 wherein the heterologous nucleic acid is at least 2 kb.

27. The method of claim 26 wherein the heterologous nucleic acid is at least 4 kb.

28. The method of claim 26 wherein the heterologous nucleic acid is at least 5 kb.

29. The method of claim 26 wherein the heterologous nucleic acid is 4-8 kb.

30. The method of claim 29 wherein the heterologous nucleic acid is 4-7.5 kb.

31. The method of claim 29 wherein the heterologous nucleic acid is 4-5 kb.

32. The method of claim 26 wherein the heterologous nucleic acid is greater than 8 kb.

33. The method of claim 20 wherein the heterologous nucleic acid encodes a T-cell receptor, a chimeric antigen receptor, or a multi-gene complex.

34. The method of claim 20 wherein the lentiviral packaging composition lacks a lentiviral tat gene.

35. The method of claim 20 further comprising a complexation solution.

36. The method of claim 35 wherein the complexation solution is OPTI-MEM or Opti-plex.

37. The method of claim 20 wherein the transfection reagent is PEIpro or PEI-MAX.

38. The method of claim 37 wherein the transfection reagent is PEIpro.

39. The method of claim 20 wherein the total plasmid mass in μg and the mass of transfection reagent in μg are present in a ratio between 1 μg total plasmid:1.0 μg of transfection reagent and 1 μg total plasmid:3.0 μg of transfection reagent.

40. The method of claim 20 wherein the total DNA concentration of gag/pol plasmid, rev plasmid, envelope plasmid, and transfer plasmid is 0.25-3.0 μg/mL.

41. The method of claim 40 wherein the total DNA concentration of the gag/pol plasmid, rev plasmid, envelope plasmid, and transfer plasmid is 0.25-2.5 μg/mL.

42. The method of claim 41 wherein the total DNA concentration of gag/pol plasmid, rev plasmid, envelope plasmid, and transfer plasmid is 1.0-2.0 μg/mL.

43. The method of claim 20 wherein the recombinant lentiviral vector is harvested at day 2-3 post-transfection.

44. The method of claim 43 wherein the recombinant lentiviral vector is harvested at day 2 post-transfection.

45. The method of claim 20 wherein the gag/pol plasmid comprises the sequence set forth at SEQ ID NO:1, the rev plasmid comprises the sequence set forth at SEQ ID NO:4, and the envelope plasmid comprises the sequence set forth at SEQ ID NO:7.
